(19) United States
(12) Patent Application Publication
(10) Pub. No.: US 2010/0331930 A1
(43) Pub. Date: Dec. 30, 2010

(54) PHOTOTHERAPY DEVICE

(76) Inventors: Thomas A.D. Burgmann, Mississauga (CA); Jackson Yu, North York (CA)

Correspondence Address: TAROLLI, SUNDHEIM, COVELL & TUMMINO L.L.P., 1300 EAST NINTH STREET, SUITE 1700, CLEVELAND, OH, 44114, US

(21) Appl. No.: 12/792,435

(22) Filed: Jun. 2, 2010

Related U.S. Application Data
(63) Continuation of application No. 11/096,443, filed on Apr. 1, 2005, now abandoned.

(51) Int. Cl. A61N 5/067; A61N 5/06

(52) U.S. Cl. 607/89; 607/88

(57) ABSTRACT

A phototherapy device includes a light source for emitting light of a suitable wavelength and a programmable controller connectable to the light source for controlling the power supplied to the light source. A programming key is associated with the programmable controller for programming the programmable controller to control the power supplied to the light source during use. The controller is programmable to control the operating configurations only when the programming key is in communication with the controller.

### FIELD OF THE INVENTION

The present invention relates generally to phototherapy and more particularly to a phototherapy device for home treatment in a variety of applications.

### BACKGROUND OF THE INVENTION

Low level light-based energy has long been used in medical applications including treatment of various disorders or conditions. Phototherapy, sometimes referred to as low level laser therapy or biostimulation, is used to externally apply low levels of light energy to tissue of a patient without thermally harming the tissue. Such phototherapy is commonly used in treatment of, for example, arthritis/rheumatism, tendonitis, repetitive stress injuries, neck and lower back pain, soft tissue injuries, migraine headaches, and acne.

Phototherapy is applied by positioning a photo-therapy light emitting device directly on the skin of the patient while gentle pressure is applied. The therapeutic light is produced by low level lasers, super luminous diodes or hyper bright light emitting diodes, and penetrates the skin and underlying tissue. Phototherapy is believed to activate enzymes in target cells, which in turn leads to protein synthesis that can enhance growth-factor response within cells and tissues and thereby stimulate natural repair processes at the cellular level.

Light energy is produced by a variety of devices such as laser diodes, light emitting diodes (LEDs) and hyper or super bright LEDs known as superluminous diodes (SLDs). Laser diodes are generally much more powerful than SLD diodes and are coherent, which results in deeper tissue penetration than SLDs. Therefore, laser diodes are used to deliver more targeted light energy to smaller treatment areas and generally in shorter time periods. SLDs are generally used in larger arrays referred to as clusters, for treating larger areas.

It has been determined that there is a range of therapeutic wavelengths of light to treat a variety of clinical conditions. The two broad categories of wavelengths most frequently used are visible (400 to 700 nm) and near-infrared (700 to 1000 nm) wavelengths. The wavelength, dosage (energy/unit area), determines the depth of penetration of the light and the therapeutic effect. Visible red light (633 nm) has a superficial penetration up to 1 cm, while infrared light (700 to 1000 nm) penetrates more deeply, ranging from 2 to 5 cm depending on the source intensity. The deeper the target tissue, the higher the dosage delivered to provide sufficient light resulting in a therapeutic effect. Thus, the wavelength and dosage are important variables in therapeutic treatment.

Many phototherapy devices exist for treatment of various conditions. One such phototherapy device is the MedX 1100 console, available through MedX Health Corp., the assignee of the present invention, 3350 Ridgeway Dr. Unit 3, Mississauga, Ontario. This device is intended for operation by a clinician or technician in a medical office or clinic. While the wavelength of the light used for therapeutic treatment is set by the light source, the dose and frequency of the light delivered is variable. These variables are set by the clinician to suitable levels for treatment of the specific condition of the patient. The clinician then applies the light to the patient for treatment until the phototherapy device completes application and the light is turned off.

While these phototherapy devices are useful for treatment of many conditions, these devices suffer disadvantages. One such disadvantage is that such phototherapy devices are located at the medical office or clinic and require a clinician or technician to set variables on the device for proper treatment of the condition. Thus, the patient must travel to the clinic or medical office for treatment. Many conditions, however, are best treated frequently, for example, on a daily basis. Thus, the patient is forced to travel to the clinic or medical office each day and await treatment by the clinician.

For convenient treatment, home devices have been proposed to allow patients to self administer phototherapy. Such devices have preset, non-adjustable doses and frequencies that are safe for sale to the general public. One such device is the Light Force Therapy LFT3000 (www.Light-Force-Therapy.com), which includes three preset selectable pulse rates. These devices suffer from disadvantages, however. One such disadvantage is that the energy dose and frequency is not optimal for the specific condition being treated. Also, the energy dose and frequency of these units cannot be adjusted as treatment progresses. Further, there is no feedback to a clinician or measurement of the effectiveness of the treatment and thus no means to adjust the treatment in response to such measurement.

It is therefore desirable to provide a phototherapy device that addresses at least some of the disadvantages of the prior art.

### SUMMARY OF THE INVENTION

In one aspect of the present invention there is provided a phototherapy device including a light source for emitting light of a suitable wavelength and a programmable controller connectable to the light source for controlling the power supplied to the light source. A programming key is associated with the programmable controller for programming the programmable controller to control the power supplied to the light source during use. The controller is programmable to control the operating configurations only when the programming key is in communication with the controller.

In another aspect of the present invention there is provided a method of programming a phototherapy device including a programmable controller and a light source connectable to the programmable controller. The method includes locating a programming key in communication with a programmable controller. The controller is programmable only when the programming key is in communication with the controller. The method also includes inputting data relating to power supplied to the light source during use and removing the programming key from communication with the programmable controller.

In yet another aspect of the present invention there is provided a programmable controller for use with a light source in phototherapy treatment. The programmable controller includes an enclosure housing a random access memory, an input interface, an output interface, a processor, a communication interface and a memory in communication over a local bus, wherein the programmable controller programmable to create or modify operating configurations only when a programming key is in communication with the programmable controller via the communication interface.

Advantageously, a phototherapy device for home treatment of a variety of conditions is provided. The phototherapy device allows for patient administered treatments set up by a clinician. The correct or desired treatment is ensured by the clinician and cannot be altered inadvertently or otherwise, by the patient. This allows for daily treatment of the condition by the patient, without requiring daily trips to a clinic or office.

Another advantage of an aspect of the present invention is the ability of the clinician to monitor and diagnose progress in treatment with the provision of outcome measures. The phototherapy device requests patients to provide feedback input by and relating to progress determined by the user. To obtain this information, the patient is prompted to answer a number of questions relating to how the patient feels at selected times. The patient provided feedback, referred to as outcome measures, is then stored and is later accessible by the clinician to monitor and diagnose progress in treatment.

### BRIEF DESCRIPTION OF THE DRAWINGS

The invention will be better understood with reference to the figures and to the following description, in which:

FIG. 1 is a perspective view of a phototherapy device according to an embodiment of the present invention;

FIG. 2 is a perspective view of a programmable controller of the phototherapy device according to the embodiment of FIG. 1;

FIG. 3 is a schematic diagram showing block elements of a programmable controller of the phototherapy device of FIG. 1; and

FIG. 4 is a perspective view of a phototherapy device including a programming key connected to the programmable controller of the phototherapy device of FIG. 1.

### DETAILED DESCRIPTION OF THE PREFERRED EMBODIMENTS

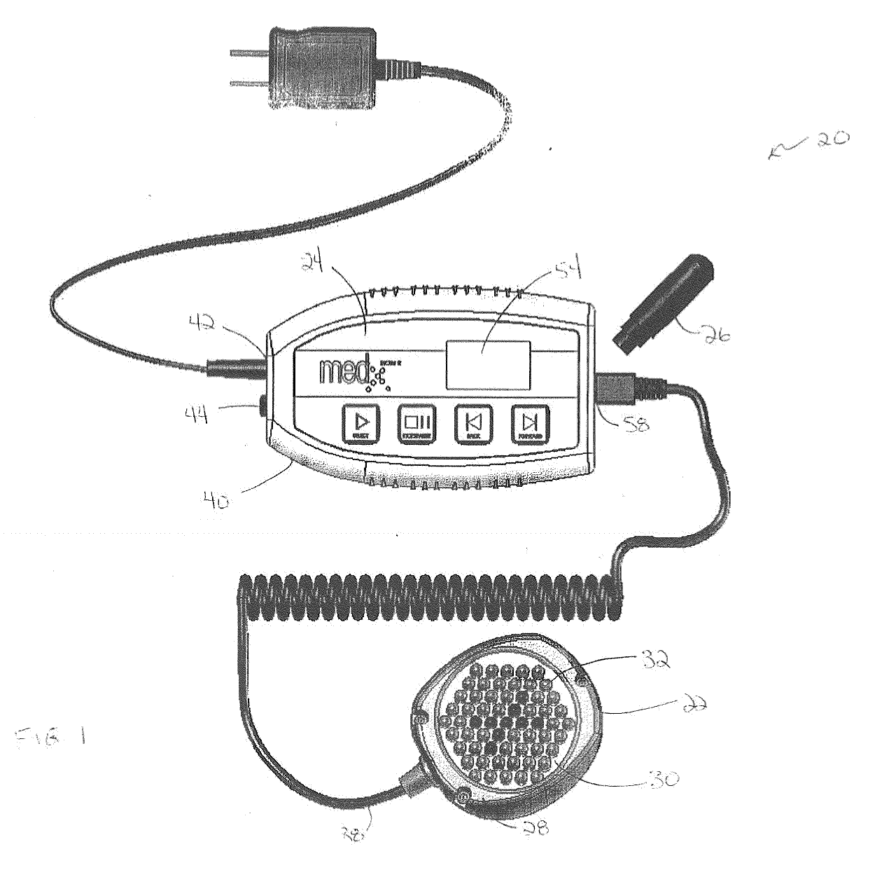

Referring first to FIG. 1, there is provided a perspective view of a phototherapy device, indicated generally by the numeral 20. The phototherapy device 20 includes a light source 22 for emitting light of a suitable wavelength and a programmable controller 24 connectable to the light source 22 for controlling the power supplied to the light source 22. A programming key 26 is associated with the programmable controller 24 for programming the programmable controller 24 to control the power supplied to the light source 22 during use. The controller 24 is programmable to control the power supplied to the light source 22 only when the programming key 26 is in communication with the controller 24.

The present invention will now be further described with continued reference to the FIG. 1. The light source 22 includes a protective housing 28 that has a generally circular footprint. All but one face of the protective housing 28 is comprised of an opaque black plastic material. The remaining face of the housing is comprised of a thin, transparent material, thereby providing a window 30, through which light is emitted. A plurality of near infrared and visible red hyper bright light emitting diodes 32 (about 870 nm and 633 nm wavelengths, respectively) are housed within the housing such that the light emitting portion of the diodes 32 face the window 30. In the present embodiment, sixty-one diodes 32 are located within the housing for emitting light through the window 30.

On the side of the housing 28 opposite the window 30, a hook side of a hook and loop type fastener adhered thereto. The hook side of the hook and loop type fastener is provided to aid in securing the housing 28 to the skin of a patient with the window 30 facing the patient's skin. This is accomplished by wrapping a loop type strap (not shown) around the patient and fixing the ends of the strap to the hook side on the housing 28. It will be understood that the strap is wrapped around the patient in a suitable location with the window 30 facing the patient's skin at the location to be treated.

A connecting cord 38 is electrically connected to the diodes 32 and extends from the housing to thereby provide electrical connection from the programmable controller 24 to the diodes 32. The opposing end of the connecting cord 38 includes a male end of a miniature DIN type connector for receipt by a port for a miniature DIN type connector in the programmable controller 24, as will be further described below.

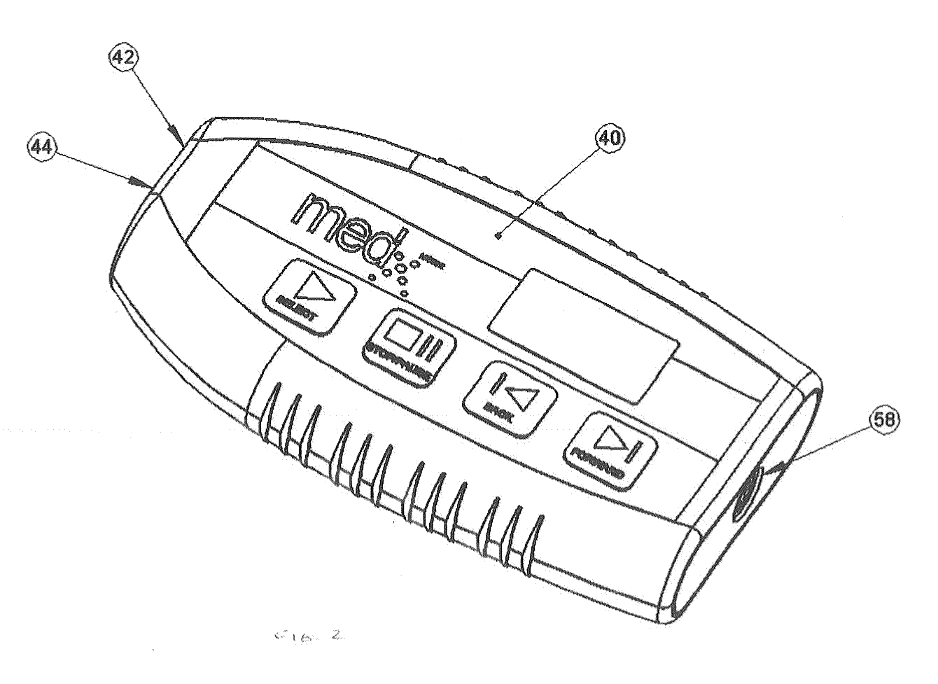

Referring now to FIG. 2, the programmable controller 24 includes an enclosure 40 in which the electronic components of the programmable controller 24 are housed. As shown, the programmable controller 24 includes an AC adapter port 42 at one side of the enclosure 40, for plugging the programmable controller 24 into a wall socket using a suitable AC adapter and providing power to the programmable controller 24. A power switch 44 for turning the controller 24 on and off is provided adjacent the AC adapter port.

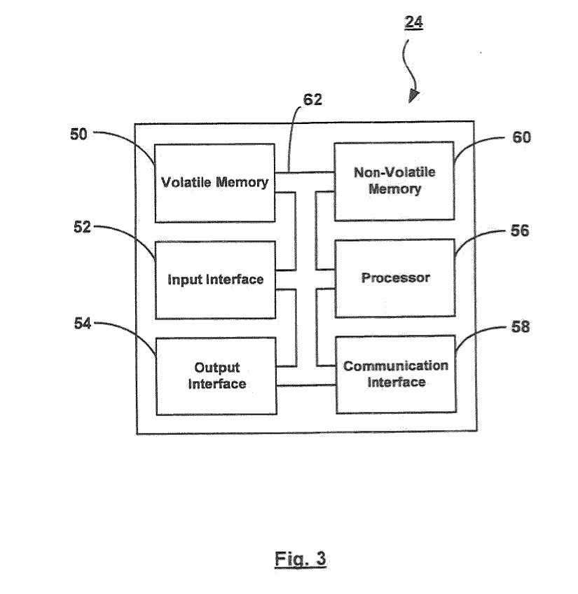

FIG. 3 is a schematic diagram showing the block elements of the electronic components of the programmable controller 24, including random access memory (“RAM”) 50, an input interface 52, an output interface 54, a processor 56, a communication interface 58 and non-volatile memory 60, all in communication over a local bus 62. The RAM 50 is volatile memory that is used to store data temporarily during operation of the programmable controller 24. The input interface 52 includes a set of buttons for controlling the operation of the programmable controller 24. Additionally, the input interface 52 allows the programming of the programmable controller 24 by a clinician or technician. The output interface 54 comprises a display for displaying configuration information and session information for the programmable controller 24. The output interface 54 also displays prompts or questions for answer by the patient to provide outcome measures which are displayed by the output interface 54 for clinician use. The non-volatile memory 60 is a flash chip that is pre-programmed with an operating system for operation of the programmable controller 24. In addition, the non-volatile memory stores a number of operating configuration numbers and operating configuration settings for operation of the programmable controller 24, operation information relating to aggregated session information, and user information including the outcome measures provided by a patient via the input interface 52. The processor 56 executes the operating system for operating the programmable controller 24. The communication interface 58 is a miniature DIN port for operating the light source 22, and for communicating with the programming key 26.

The operating system enables the programmable controller 24 to be conditioned between three modes: a user input mode, an operating mode and a programming mode. In the user input mode, the operating system presents questions or prompts soliciting user input, and registers outcome measures in the form of user input provided in response to the questions or prompts, in the non-volatile memory 60.

In the operating mode, the operating system presents operating information, session information and details of an active operating configuration number and designation letter on the output interface 54. Additionally, the operating system allows for control of the operation of the programmable controller 24 including selection of a designation letter associated with an active operating configuration number.

In the programming mode, the operating system enables a clinician to modify the operating configurations. During the programming mode, the operating system presents details of a selected operating configuration number via the output interface 54, and registers input received via the input interface 52, from the clinician for modifying the details of the selected operating configuration number. The operating system also enables the clinician to select up to five operating configuration numbers as active operating configuration numbers by associating up to five operating configuration numbers with a respective letter designation for controlling operation of the programmable controller 24.

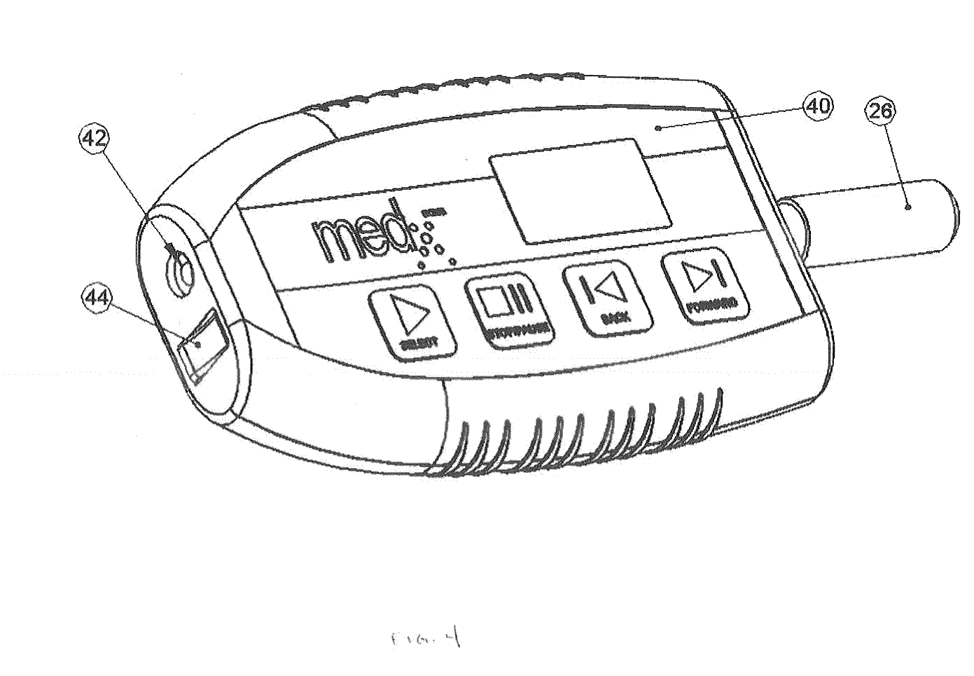

The programming key 26 for connection to the communication interface 58 of the programmable controller 24 is shown in FIG. 4. The programming key 26 includes a unique fixed resistor connected to an ID pin. When the programming key 26 is connected to the communication interface 58, the resistance is measured by the programmable controller 24, which identifies the programming key 26 by matching the measured resistance to a look-up table stored in the non-volatile memory 60. When the programming key 26 is inserted into the communication interface 58 of the programmable controller 24, the programmable controller 24 identifies the programming key 26 and the programmable controller 24 enters the programming mode.

In use, the programmable controller 24 is plugged into a standard 100-120V 50/60 Hz outlet using a suitable AC adapter and cord connected to the AC adapter port 42. Prior to activating the programmable controller 24 using the power switch 44, the programming key 26 is inserted into the communication interface 58 of the programmable controller 24, thereby causing the programmable controller 24 to enter the programming mode. Note that the programmable controller 24 does not enter the programming mode without insertion of the programming key 26 into the communication interface 58. After inserting the programming key 26, the power switch 44 is turned on to activate the programmable controller 24.

When turned on with the programming key 26 inserted into the communication interface 58, the clinician is prompted to select operating configurations for operation of the programmable controller 24, aggregated session information and user information previously provided by an end-user or patient, by prompts appearing on the output interface 54. The operating configurations include operating configuration numbers and active operating configuration numbers designated by respective letters, each operating configuration number with settings for therapy treatments, including dosage (Joules/cm²), pulse frequency (Hz) and duty cycle (percentage of time on). When the operating configurations are selected using the input interface 52, the clinician is prompted to select an operating configuration number using the input interface 52 and for the configuration number selected, the clinician is prompted to enter or modify the settings including dosage, pulse frequency and duty cycle. The clinician is able to enter or modify settings for multiple operating configuration numbers and to set up to five operating configuration numbers as active operating configuration numbers by associating each operating configuration number with a respective letter designation. It will be appreciated that the operating configuration numbers selected as active operating configuration numbers are programmed by the clinician with the appropriate operating configuration settings for treatment of the condition or conditions of the patient. Each of the operating configuration numbers and operating configuration letter designations associated with operating configuration numbers are stored in the non-volatile memory 60.

When the aggregated session information is selected, aggregated session information is displayed on the output interface 54. This information includes, for example, the number of times the patient treated themselves and cumulative treatment time.

When the user information previously provided by an end-user or patient is selected, the user information is displayed on the output interface, including outcome measures input by the patient to assist the clinician in monitoring and diagnosing progress of treatment. In the present embodiment, outcome measures are input by the patient for monitoring by the clinician. These outcome measures include one or more of, for example, patient estimated ratings of pain, range of motion, activity, stiffness, sensation, sleep, work and enjoyment of life.

To exit the programming mode, the power switch 44 is turned off to deactivate the programmable controller 24 and the programming key 26 is removed from the communication interface 58.

For therapeutic treatment, the light source 22 is connected to the programmable controller 24 by connecting the miniature DIN connector of the connecting cord 38 into the communication interface 58 of the programmable controller 24. With the light source 22 connected to the programmable controller 24, the programmable controller 24 enters the user input mode, in which the operating system presents questions or prompts on the output interface 54, to the patient and the patient provides outcome measures in the form of answers to the questions or prompts, using the input interface 52. As previously indicated, one or more outcome measures are input by the patient, including, for example, patient estimated ratings of pain, range of motion, activity, stiffness, sensation, sleep, work and enjoyment of life. The outcome measures are then stored in the non-volatile memory 60. After the patient provides each of the outcome measures, the programmable controller 24 enters the operating mode.

In the operating mode, the operating system presents operating information, session information and details of active operating configuration numbers designated by respective letters, on the output interface 54. The patient is then able to select a letter designation that is associated with an operating configuration number as previously selected by the clinician when in the programming mode. After selection of the letter designation, treatment is started by the patient by controlling the programmable controller 24 using the input interface 52. Thus, light is emitted from the light source 22 and is controlled to provide phototherapy at the previously programmed settings for the operating configuration number associated with the selected designation letter. It will be appreciated that different operating configuration settings for different operating configuration numbers set as active with respective letter designations are useful to allow the patient to choose different letter designations for treatment of, for example, different conditions for a single patient or for different treatments as healing of the condition progresses. The therapy treatment is controlled by the patient by stopping, pausing or beginning the treatment using the input interface 52.

Prior to beginning treatment, the patient self-applies the window 30 of the light source 22 to their skin at the desired treatment location, either by hand or by securing the light source 22 to the patient using a loop type strap (not shown).

### Exemplary Treatments

The following are examples that are provided to further illustrate various embodiments of the present invention. These examples are intended to be illustrative only and are not intended to limit the scope of the present invention.

In treatment of carpal tunnel syndrome, the programming key 26 in inserted into the communication interface 58 and the programmable controller 24 is turned on using the power switch 44. The clinician selects operating configurations and is prompted to select an operating configuration number for the programmable controller 24, using the input interface 52. In the present embodiment, the clinician selects a manual operating configuration number m1. The operating configuration settings for the operating configuration number m1 is then set by selecting the dosage, pulse frequency and duty cycle. For treatment of carpal tunnel syndrome, the dosage is set to a level of about 8 to about 10 Joules/cm². The pulse frequency is set to zero, thereby making the duty cycle 100% to be delivered. Next, the operating configuration number m1 is set as active by selecting designation letter A in association with operating configuration number m1. The patient is then instructed in proper technique for treatment 1 to 2 times per day depending on the patient condition.

In use, the patient connects the light source 22 to the programmable controller 24, turns the programmable controller 24 on and the programmable controller 24 enters the user input mode. The patient is then prompted to provide outcome measures, including patient estimated ratings that are then stored in the non-volatile memory 60. After the patient provides each of the outcome measures, the programmable controller 24 enters the operating mode.

In the operating mode, the programmable controller 24 automatically enters operating configuration number m1 associated with designation letter A since only designation letter A is programmed to be associated with an operating configuration number m1. Thus, only configuration designation letter A is chosen. The light source 22 is then placed over the patient's skin at the treatment site as instructed by the clinician and the patient initiates treatment using the input interface 52.

When the programmable controller 24 is returned to the clinician, the programming key 26 is inserted into the communication interface 58 of the programmable controller 24 and the programmable controller 24 is turned on using the power switch 44. The clinician then selects aggregated session information and the aggregated session information, including the number of times the patient treated themselves, the total treatment time and the outcome measures input by the patient, is displayed on the output interface 54. The information is presented in a manner that allows the clinician to scroll through all of the information.

In treatment of arthritis of the hand and knee, the programming key 26 in inserted into the communication interface 58 and the power switch 44 is switched to the on position. The clinician then selects operating configurations for the programmable controller 24 and the clinician is then prompted to select an operating configuration number using the input interface 52. In the present embodiment, the clinician selects manual operating configuration number m1 and the operating configuration settings for the operating configuration number m1 are then set by selecting the dosage, frequency and duty cycle. For treatment of the hand, the dosage is set to a level of about 6 to about 8 Joules/cm². The frequency is set to a pulse frequency of 0 and the duty cycle is thereby set to 100% to be delivered. Next, the operating configuration number m1 is set as active by selecting designation letter A in association with operating configuration number m1. The patient is then instructed in proper technique for treatment 1 to 2 times per day depending on the patient condition. The manual operating configuration number m2 is then selected and the operating configuration settings for the operating configuration number m2 are set by selecting dosage, frequency and duty cycle. For treatment of the knee, the dosage is set to a level of about 8 to about 10 Joules/cm². The frequency is set to 10 to 20 Hz and the duty cycle is set to 80% to be delivered. Next, the operating configuration number m2 is set as active by selecting designation letter B is association with operating configuration number m2. The patient is then instructed in proper administration for treatment 1 to 2 times per day.

In use, the patient connects the light source 22 to the programmable controller 24, turns the programmable controller 24 on and the programmable controller 24 enters the user input mode. The patient is then prompted to provide outcome measures, first relating to designation letter A (for the hand arthritis) and then relating to designation letter B (for the knee arthritis). Again, the outcome measures are stored in the non-volatile memory 60. After the patient provides each of the outcome measures, the programmable controller 24 enters the operating mode.

In the operating mode, the programmable controller 24 automatically enters operating configuration 1 and the patient is given the choice between configuration designation letter A and configuration designation letter B. When A is chosen, the programmable controller enters operating configuration number m1, the light source 22 is then placed over the patient's skin at the treatment site for the hand as instructed by the clinician and the patient initiates treatment using the input interface 52. When B is chosen, the programmable controller enters operating configuration number m2, the light source 22 is then placed over the patient's skin at the treatment site for the knee as instructed by the clinician and the patient initiates treatment using the input interface 52.

When the programmable controller 24 is returned to the clinician, the programming key 26 is inserted into the communication interface 58 of the programmable controller 24 and the programmable controller 24 is turned on using the power switch 44. The clinician then selects aggregated session information and the aggregated session information for each configuration designation letter, including the number of times the patient treated themselves, the total treatment time and the outcome measures input by the patient, is displayed on the output interface 54. The information is presented in a manner that allows the clinician to scroll through all of the information.

A specific embodiment of the present invention has been shown and described herein. However, modifications and variations to this embodiment are possible. For example, the size and shape of the components may vary while still performing the same function. For example, the shape of the light source 22 can be any shape rather than round. Rather than only light emitting diodes, the light source can include solid state lasers. Also, the outcome measures may differ from those discussed above. Those skilled in the art may conceive of still other modifications and variations, all of which are within the scope and sphere of the present invention.

1-35. (canceled)

36. A phototherapy device comprising:
a light source configured to emit light of a suitable wavelength;
a programmable controller, in an operating mode said programmable controller being configured to control operation of said light source in accordance with at least one operating configuration stored in memory therein; and
a programming key removably insertable into a port of said programmable controller, said programmable controller being configured to automatically enter a programming mode when said programming key is inserted into said port, in said programming mode said programmable controller being configured to alter the at least one operating configuration stored in said memory in response to user input entered via said programmable controller.

37. The phototherapy device of claim 1, wherein said light source comprises a housing accommodating one of a plurality of light emitting diodes, a plurality of solid state lasers and a combination of light emitting diodes and solid state lasers.

38. The phototherapy device of claim 2, wherein said light source communicates with said programmable controller via said port.

39. The phototherapy device of claim 1, wherein a plurality of operating configurations is stored in said memory.

40. The phototherapy device of claim 4, wherein said programmable controller further comprises a plurality of input keys.

41. The phototherapy device of claim 5, wherein said programmable controller further comprises a display configured to display information during programming or use.

42. The phototherapy device of claim 4, wherein each of said operating configurations comprises light dosage and light pulse frequency settings.

43. The phototherapy device of claim 7, wherein in said operating mode the programmable controller is further configured to track and store usage information.

44. The phototherapy device of claim 1, further comprising attachment structure on said housing, said attachment structure being configured to attach said housing to a patient.

45. The phototherapy device of claim 5, wherein said programmable controller is further configured to enter an input mode, in said input mode said programmable controller being configured to prompt a user to input treatment progress information

46. The phototherapy device of claim 10, wherein the treatment progress information comprises estimates of any of pain, range of motion, activity, stiffness, sensation, sleep, work, and enjoyment of life.

47. The phototherapy device of claim 1 wherein said programming key comprises a unique identifier readable by said programmable controller.

48. A phototherapy device comprising:
a light source;
a programmable controller communicating with said light source, said light source being removeably connectable to a port of said programmable controller, said programmable controller being configured to control operation of said light source in accordance with program instructions stored in memory therein when said light source is connected to said port; and
a programming key removeably connectable to said port, said programmable controller entering a programming mode only when said programming key is connected to said port, in said programming mode said programmable controller being configured to alter the program instructions stored in said memory in response to user input entered via said programmable controller.

49. The phototherapy device of claim 13, wherein said light source comprises a housing accommodating a plurality of light emitting diodes.

50. The phototherapy device of claim 14, wherein said light emitting diodes comprise infrared and visible red light emitting diodes.

51. The phototherapy device of claim 14, further comprising attachment structure on said housing, said attachment structure being configured to attach said housing to a patient.

52. The phototherapy device of claim 13, wherein said programmable controller further comprises a plurality of input keys.

53. The phototherapy device of claim 17, wherein said programmable controller further comprises a display configured to display information during programming or use.

54. The phototherapy device of claim 17, wherein said program instructions comprise settings for light dosage, light pulse frequency, and duty cycle.

55. The phototherapy device of claim 17, wherein said programmable controller is further configured to enter an input mode, in said input mode said programmable controller being configured to prompt a user to input treatment progress information

56. The phototherapy device of claim 13, wherein said programmable controller is configured to automatically enter said programming mode upon insertion of said programming key into said port.
